(19) United States
(12) Patent Application Publication
(10) Pub. No.: US 2011/0151549 A1
(43) Pub. Date: Jun. 23, 2011

(54) Device For Carrying Out An Individual Immunoassay In A Fully Automatic Manner

(76) Inventors: Wigbert Berg, Mainz (DE); Stephan Becker, Buttelborn (DE)

(21) Appl. No.: 11/719,060

(22) PCT Filed: Nov. 10, 2005
(86) PCT No.: PCT/EP2005/012060

§ 371 Date: Nov. 20, 2007

(30) Foreign Application Priority Data
Nov. 11, 2004 (DE) 10 2004 054 551.0

(51) Int. Cl. G01N 33/558; C12M 1/34

(52) U.S. Cl. 435/287.2; 436/514

(57) ABSTRACT

The invention relates to a device for carrying out an individual immunoassay in a fully automatic manner in order to detect the presence of a biologically active substance in a sample, and to the use of one such device. The invention also relates to a sample strip which, in order to carry out an individual immunoassay for the detection of a biologically active substance in a sample using the above-mentioned device, comprises a plurality of cavities for providing the reagents required for the assay. The sample strip is sealed with a film carrying a code specific to the assay.

The invention relates to a device for carrying out an individual immunoassay in a fully automatic manner in order to detect the presence of a biologically active substance in a sample, and also to the use thereof. In addition, the invention relates to a sample strip for carrying out an individual immunoassay in order to detect the presence of a biologically active substance in a sample using the above-mentioned device, and also to a kit comprising this sample strip.

Immunoassays, which are used for determining biologically active substances such as antigens, antibodies or haptens, are nowadays widespread in a broad range of technical fields such as, for example, medical diagnosis. To determine these biologically active substances, immunoassays use, in particular, the highly specific antigen/antibody reaction. In order to be able to comment both qualitatively and quantitatively on the antigen/antibody reaction, one of the reactants is coupled to a readily detectable labelling substance in such a way that the immunological properties of the components are substantially maintained. Suitable labelling substances include radioisotopes (“radioimmunoassay”) or else enzymes and the associated substrates (enzyme immunoassays such as, for example, ELISA). However, on account of their advantageous properties, enzymes and the associated substrates are preferably used as a labelling system in what is known as the enzyme immunoassay. In this case, the antigen/antibody reaction is then linked to an enzymatic reaction, use being made of either antibody/enzyme or antigen/enzyme conjugates which bind to the biologically active substances to be detected and are determined photometrically or fluorimetrically, after addition of a suitable substrate, by measuring the enzyme activity of the conjugate. The catalytic effect of the enzyme also intensifies the measuring effect, as although the antibody/enzyme or antigen/enzyme conjugates bind stoichiometrically to the biologically active substances to be detected, the enzyme of the coupled conjugate can convert not just one substrate but rather a large number of substrates during the course of the detection reaction. However, this means that for each immunoassay to be carried out, calibration must initially take place with a number of samples (calibrators) having various known concentrations.

Nowadays, immunoassays are conventionally carried out in fully automatic immunoassay machines. These fully automatic immunoassay machines operate with 96-well microtitre plates containing 96 cavities for samples and the calibration of the immunoassay to be carried out. These microtitre plates generally have 90 cavities for samples and six cavities for the calibration of the immunoassay. For the calibration of the immunoassay, there is to be produced a calibration curve using calibrators having known concentrations, which curve generally comprises five to six points and the validity of which is additionally ensured using two further control substances. The immunoassay in the fully automatic immunoassay machines comprising the samples, calibrators and control substances located in the cavities in the microtitre plate is carried out in accordance with the following model:
diluting the samples, calibrators and control substances,
incubating the samples, calibrators and control substances in the cavities coated with a selective binding partner of the biologically active substance to be detected (for example, antigens or antibodies), so the biologically active substance to be detected can be bound,
washing-out of non-bound constituents of the sample,
addition of a detection reagent, preferably an enzyme-labelled antibody or antigen, which binds to the bound biologically active substance to be detected,
washing-out of non-bound detection molecules and
addition of a substrate, the colour of which is converted proportionally to the presence of the detection reagent.

The reagents required for the immunoassay, such as for example the buffers required for diluting the samples, calibrators and control substances and also the reagents for washing and for the detection reaction, are provided in large storage containers in the fully automatic immunoassay machines.

However, the fully automatic immunoassay machines which are currently commercially available have various drawbacks. A fundamental drawback is that all of the samples located on a 96-well microtitre plate have to be subjected to the same immunoassay, i.e. are examined for the same parameter. This greatly restricts the required flexibility in the diagnostics of the laboratory to be implemented, so the use of a fully automatic immunoassay machine is cost-effective only if all 96 cavities in the microtitre plate are utilised within a test series having a relatively high number of samples. However, in various situations, this leads to problems which have a detrimental effect on the analysis and on the owner of the sample to be analysed such as, for example, a patient. Thus, in medical emergencies, for example, individual parameters or a group of various parameters have to be determined very rapidly. However, for financial reasons, this is carried out manually rather than in a fully automatic manner, and this entails a greater risk of false results than if the immunoassay were carried out in a fully automatic manner. In addition, small and medium-sized laboratories and clinics very often cannot combine the samples from patients to form such large test series and have, on account of the insufficient utilisation of the fully automatic immunoassay machine, either to forward the samples to larger laboratories or to accumulate them over a relatively long period of time before the immunoassay is carried out. This results in an unacceptable loss of time. In addition, for some important clinical parameters such as for example for ganglioside antibodies, there has previously not been an automated immunoassay simply because these parameters occur very rarely and are therefore unsuitable for an immunoassay in a fully automatic immunoassay machine comprising 96-well microtitre plates.

Individual manufacturers of fully automatic immunoassay machines have recently attempted, by developing new equipment, to eliminate at least some of the drawbacks of the fully automatic immunoassay machines, comprising 96-well microtitre plates.

DPC Biermann, for example, offers an immunoassay analyser (IMMULITE®) which is able to carry out individually a specific immunoassay (“individual immunoassay”) for each sample to be examined. Like the above-described fully automatic immunoassay machines, inside this device are accommodated in large storage containers all of the reaction reagents and washing solutions required for each immunoassay to be carried out, whereas the solid phase for the immunoassay is provided in the form of beads in a sealed test kit. However, the provision in large storage containers in the device of all of the reagents and washing solutions required for each immunoassay to be carried out entails the drawback that these reagents and washing solutions have constantly to be exchanged in the storage containers, should various immunoassays have to be carried out in succession, as storage containers in the device have a limited capacity. The device supplied by DPC Biermann also has the drawback of requiring relatively high sample volumes, on account of the samples and test tubes used, and this is problematic in the field of pediatrics, for example, in which autoimmunological issues frequently arise. In order to simplify the calibration of each individual immunoassay to be carried out using the immunoassay analyser, calibration curves for common immunoassays are entered in the evaluation program of the device. However, this resulted in low flexibility in the use of the immunoassay analyser, as on the introduction of new tests, calibration curves have first to be newly entered in the device or a calibration curve has to be produced for each test to be carried out. Furthermore, the immunoassay analyser from DPC Biermann has a relatively high purchase price, so this device would not appear to be suitable for small and medium-sized laboratories and clinics, in particular.

Moreover, BIOMÉRIEUX offers an immunoassay analyser which is also suitable for individually determining a sample using a specific immunoassay. The reagents required for each immunoassay to be carried out are provided in a test-specific kit, whereas the required washing solution is located in a storage container in the device. The reaction chamber in which each immunoassay is carried out is located in an additional kit. This increases, firstly, the complexity of controlling the device and, secondly, the operational complexity and the error-proneness for carrying out the immunoassay. In order to facilitate the calibration of the individual immunoassays to be carried out, a calibration curve is stored in the test-specific kit for each immunoassay. However, a drawback of the immunoassay analyser sold by BIOMÉRIEUX is that this system is still restricted to a small number of applications in assay technology and internal measurements have revealed that the system displays major problems in the reproducibility of results. From the point of view of price, too, the immunoassay analyser from BIOMÉRIEUX would not appear to be a particularly attractive prospect to small and medium-sized laboratories and clinics.

In summary, there has to date been no known suitable system for carrying out an individual immunoassay in a fully automatic manner in order to detect the presence of a biologically active substance, such as for example an antibody, an antigen or a hapten, which system allows an immunoassay to be carried out in a simple, flexible, rapid and reproducible manner using a single sample, requiring little space, having low purchase costs and also low operating and maintenance costs, and is suitable, in particular, for autoimmune diagnosis.

The aim of the present invention was therefore to provide a system for carrying out an individual immunoassay in a fully automatic manner in order to demonstrate the presence of a biologically active substance, which system meets the above-mentioned requirements.

According to the invention, this object is achieved by a device for carrying out an individual immunoassay in a fully automatic manner in order to detect the presence of a biologically active substance in a sample, comprising

(a) a means of receiving at least one sample strip, the sample strip comprising in an exclusively linear arrangement at least five cavities, of which at least one cavity (i) serves to provide the sample to be examined, at least one further cavity (ii) serves as a reaction chamber for carrying out the individual immunoassay, at least one further cavity (iii) serves as a reaction reagent reservoir for providing the reaction reagent(s) required for the individual immunoassay and optionally at least one further cavity (iv) serves to provide a control substance,

(b) a means for conveying the at least one sample strip along a transfer route to different reaction stations of the individual immunoassay, wherein the reaction stations comprise at least one decoding station, at least one pipetting station, at least one washing station and at least one detection station,

(c) at least one means for decoding a code specific to the individual immunoassay at the at least one decoding station,

(d) at least one pipetting system for carrying out the reaction steps of the individual immunoassay that are to be carried out at the at least one pipetting station and/or the at least one washing station,

(e) at least one reservoir for providing the washing and/or rinsing solution required at the at least one pipetting station and/or the at least one washing station,

(f) at least one means for evaluating the individual immunoassay at the at least one detection station, and

(g) at least one means for controlling the individual immunoassay in a fully automatic manner.

The device according to the invention is suitable for carrying out an individual immunoassay in a fully automatic manner in order to detect the presence of any desired biologically active substance in a sample such as, for example, an antibody, an antigen, a hapten, a hormone, a pharmacon, an opiate and also a diagnostically important protein. Preferably, the device is suitable for carrying out an individual immunoassay in a fully automatic manner in order to detect the presence of an antibody or antigen in a sample. Particularly preferably, the antibody to be detected using the individual immunoassay is an autoimmune antibody. Suitable autoimmune antibodies which can be detected using the device according to the invention are well known to a person skilled in the art.

The device according to the invention, which is suitable for carrying out an individual immunoassay in a fully automatic manner in order to detect the presence of a biologically active substance in a sample, comprises a means (a) for receiving at least one sample strip. Preferably, the means (a) can receive up to 30 sample strips, i.e. 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 sample strips. In a particularly preferred embodiment of the present invention, the means (a) is a rotary table. According to the invention, the rotary table is configured so as to be able securely and stably to receive the individual sample strips. For example, the rotary table has recesses for receiving the individual sample strips, in which recesses the samples strips are held securely and stably throughout the individual immunoassay.

According to the invention, the at least one sample strip intended to be received in the means (a) of the device according to the invention has in an exclusively linear arrangement at least five cavities, of which at least one cavity (i) serves to provide the sample to be examined, at least one further cavity (ii) serves as a reaction chamber for carrying out the individual immunoassay, at least one further cavity (iii) serves as a reaction reagent reservoir for providing the reaction reagent(s) required for the individual immunoassay and optionally at least one further cavity (iv) serves to provide a control substance. According to the present invention, the term “in an exclusively linear arrangement” means that all of the cavities located on the sample strip are located exclusively in a linear arrangement relative to one another.

The at least one cavity (i) of the sample strip serves to provide the sample to be examined. In a preferred embodiment of the present invention, the at least one cavity (i) tapers toward the base in order to allow an individual immunoassay to be carried out even at very low sample volumes.

The at least one cavity (ii) of the sample strip serves as a reaction chamber for carrying out the individual immunoassay. Preferably, the at least one cavity (ii) of the sample strip is coated, as a function of the individual immunoassay to be carried out, with a suitable binding partner which, under the reaction conditions of the individual immunoassay to be carried out, can bind to the biologically active substance to be detected. According to the invention, the binding partner is selected, as a function of the individual immunoassay to be carried out and the biologically active substance to be detected, from antigens, antibodies, receptors, substrates and substrate analogues, inhibitors, cofactors, etc.

If the device serves to carry out an individual immunoassay in a fully automatic manner in order to detect the presence of an antibody, the binding partner is the corresponding antigen to the antibody to be detected.

Furthermore, the at least one cavity (iii) of the sample strip serves as a reaction reagent reservoir for providing the reaction reagent(s) required for the individual immunoassay. According to the present invention, the at least one cavity (iii) serves to provide all of the reaction reagents required for the individual immunoassay except for the washing and/or rinsing solution required to carry out the individual immunoassay, each individual reaction reagent being located in a separate cavity (iii). The reaction reagent(s) required for the individual immunoassay can, for example, be a suitable buffer for diluting the sample to be examined or a control substance, and also one or more detection reagents for the detection reaction. Preferably, the detection reagent according to the present invention is selected from an enzyme-labelled antigen or antibody which binds to the biologically active substance to be detected and an associated, readily detectable substrate, the conversion of which by the enzyme of the enzyme-labelled antigen or antibody can be monitored photometrically or fluorimetrically. Exemplary enzyme-labelled antigens or antibodies and the associated substrates comprise peroxidase-labelled antigens or antibodies and also hydrogen peroxide as the substrate. Further enzyme-labelled antigens or antibodies and the associated substrates are well known to a person skilled in the art.

The at least one cavity (iv) optionally provided in the sample strip serves to provide a control substance. The control substance is selected, as a function of the individual immunoassay to be carried out, from substances such as antibodies, antigens, proteins, etc. and serves to check and compensate for measurement fluctuations in the individual immunoassay to be carried out.

In a preferred embodiment of the invention, the at least one sample strip intended to be received in the means (a) of the device according to the invention has in an exclusively linear arrangement at least seven cavities, particularly preferably eight or twelve cavities, of which one cavity (i) serves to provide the sample to be examined, two cavities (ii) serve as a reaction chamber for carrying out the individual immunoassay with the sample to be examined and also with a control substance, three cavities (iii) serve as a reaction reagent reservoir for providing the reaction reagents required for the individual immunoassay and one cavity (iv) serves to provide the control substance. Preferably, a first cavity (iii) serves to provide a sample dilution buffer, a second cavity (iii) serves to provide a first detection reagent, such as an enzyme-labelled antigen or antibody, and a third cavity (iii) serves to provide a second detection reagent such as a substrate for detection of the enzyme-labelled antigen or antibody bound to the biologically active substance to be detected.

In a further preferred embodiment of the invention, the sample strip intended to be received in the means (a) of the device according to the invention is a longitudinal or transverse strip of a 96-well microtitre plate comprising twelve or eight cavities.

Moreover, according to a particularly preferred embodiment of the invention, the sample strip is sealed with a film. Preferably, the film has one or more perforations, so it can easily be removed at least in part, i.e. in the region of individual cavities, before the sample strip is received in the means (a). More preferably, the film has one or more perforations, in such a way that the film can be removed via the cavities (i) and/or (ii) before the sample strip is received in the means (a). If the detection reaction of the individual immunoassay is in the form of a colour reaction, it is crucial that the film be removed via the cavities (ii) in order to ensure an accurate reading of the colour reaction by the device according to the invention. However, all of the other cavities should remain sealed with the film in order to prevent unnecessary contamination of the reaction reagents and the control substance. Furthermore, the present invention provides for the film optionally also to have a code specific to the individual immunoassay. Preferably, the code specific to the individual immunoassay is located on a region of the film that is not removed before the sample strip is received in the means (a). More preferably, the code specific to the individual immunoassay is located above the cavities (iii) and (iv). In this case, the film is of a composition allowing the pipetting system to pierce the film during the individual immunoassay. According to the present invention, the code specific to the individual immunoassay is used, for example, for automatic detection by the device according to the invention of the individual immunoassay to be carried out and/or storage of the calibration curve of the individual immunoassay to be carried out. The code specific to the individual immunoassay can be any code with which a person skilled in the art is familiar. Preferably, the code specific to the individual immunoassay is a bar code.

The device according to the invention further comprises a means (b) for conveying the at least one sample strip along a transfer route to different reaction stations of the individual immunoassay. Preferably, the means (b) comprises a motor for controlling the means (a) for receiving the at least one sample strip, allowing the at least one sample strip received by the means (a) purposefully to be conveyed to the different reaction stations of the individual immunoassay. According to the invention, the different reaction stations of the individual immunoassay comprise at least one decoding station in order to decode a code specific to the individual immunoassay and optionally to start the carrying-out of the individual immunoassay in a fully automatic manner, at least one pipetting station in order to add to the reaction mixture and optionally then to remove again the reaction reagents required for the individual immunoassay, at least one washing station in order to remove non-bound substances during the individual reaction steps of the individual immunoassay and to rinse the pipetting system of the at least one pipetting station, and at least one detection station in order to detect the biologically active substance to be detected and computationally to process the obtained measured values. More preferably, the different reaction stations of the individual immunoassay comprise a decoding station, a first pipetting station in order to transfer into the cavities (ii) and, if necessary, to dilute with a dilution buffer the sample to be examined and optionally a control substance, a first washing station in order to wash out non-bound constituents of the sample and optionally of the control substance, a second pipetting station in order to transfer a detection reagent into the cavities (ii) and a detection station. If the detection reagent consists of an enzyme-labelled antigen or antibody and the associated substrate, the different reaction stations of the individual immunoassay additionally comprise, after the second pipetting station, a second washing station in order to wash out non-bound, enzyme-labelled antigen or non-bound, enzyme-labelled antibodies and a third pipetting station in order to transfer the substrate into the cavities (ii).

The device according to the invention further comprises at least one means (c) for decoding a code specific to the individual immunoassay at the at least one decoding station. Conventional systems for decoding a code which is specific to the individual immunoassay and attached to the sample strip are well known to a person skilled in the art. The means (c) is preferably a conventional system for the decoding of bar codes.

The device according to the invention also comprises at least one pipetting system (d) for carrying out the reaction steps of the individual immunoassay that are to be carried out at the at least one pipetting station and/or the at least one washing station.

The device according to the invention also comprises at least one reservoir (e) for providing the washing and/or rinsing solution required at the at least one pipetting station and/or the at least one washing station. Whereas the remaining reaction reagents required for each individual immunoassay to be carried out are provided in the sample strip, the present invention provides for the washing and/or rinsing solution, which can be used for a broad range of individual immunoassays which can be carried out using the device, to be provided for the sake of simplicity in a reservoir in the device and not on the sample strip.

The device according to the invention further comprises at least one means (f) for evaluating the individual immunoassay at the at least one detection station. According to the present invention, the at least one means (f) is selected from a photometer, a fluorimeter, a computer with associated software, etc. The device according to the invention preferably has at least two means (f) for evaluating the individual immunoassay, including a photometer or fluorimeter, with which, for example, the conversion of the substrate by the enzyme-labelled antigen or the enzyme-labelled antibody can be monitored photometrically or fluorimetrically, and a computer with associated software which also undertakes an evaluation of the measured data while taking into account a matching with the calibration curve stored on the sample strip and also the optionally measured control values.

Finally, the device according to the invention comprises at least one means (g) for controlling the individual immunoassay in a fully automatic manner. The means (g) is preferably a computer with associated software. More preferably, the means (g) is the computer which may be present at the at least one detection station.

The main advantage of the device according to the invention over existing technologies consists in the concentration of the individual immunoassay as a whole on the optionally sealed sample strip. This strip contains all of the reaction reagents except for the washing and/or rinsing solution and also the solid phase of the individual immunoassay. Also stored on the sample strip is the calibration curve required for evaluating the individual immunoassay. The present invention thus provides an extremely user-friendly and low-maintenance immunoassay analyser which allows an immunoassay to be carried out in a simple, flexible, rapid, and reproducible manner using a single sample, an analyser which requires little space and is particularly suitable for autoimmune diagnostics.

The invention further relates to the use of the device according to the invention for carrying out an individual immunoassay in a fully automatic manner in order to detect the presence of a biologically active substance in a sample, including the steps:
(a) preparing the at least one sample strip,
(b) inserting the at least one sample strip into the device,
(c) transferring the sample from the at least one cavity (i) into a cavity (ii) and optionally a control substance from the at least one cavity (iv) into a further cavity (ii),
(d) incubating the sample and optionally a control substance in the cavity/cavities (ii),
(e) washing the cavity/cavities (ii) with washing and/or rinsing solution stored in the device,
(f) transferring detection reagents stored in the sample strip from the at least one cavity (iii) into the cavity/cavities (ii), and
(g) detecting the biologically active substance to be detected.

The use according to the invention includes in step (a) the preparing of the at least one sample strip. The preparing of the at least one sample strip includes the coating of the at least one cavity (ii) with a suitable binding partner for the biologically active substance to be detected, the introducing of the sample to be examined into the at least one cavity (i) in the sample strip, the introducing of the reaction reagents required for the individual immunoassay to be carried out, except for the required washing and/or rinsing solution, into the at least one cavity (iii) in the sample strip and optionally the introducing of a control substance into the at least one cavity (iv) in the sample strip. In a preferred embodiment of the invention, the sample strip to be prepared is a sample strip which is sealed with a film and of which at least one cavity (ii) is already coated with a suitable binding partner and of which at least one cavity (iii) and optionally at least one cavity (iv) already contain the reaction reagent(s) required for the individual immunoassay to be carried out and optionally a control substance. The present invention provides in this regard for the preparing of the at least one sample strip to include the removing of the film of the sealed sample strip at least in the region of the at least cavity (i) and/or the at least one cavity (ii) and also the introducing of the sample to be examined into the at least one cavity (i) in the sample strip. In the region of the other cavity and, in particular, in the event of the film having in the region of the at least one cavity (iii) and (iv) a code specific to the individual immunoassay, the sample strip can remain sealed with the film during the carrying-out of the individual immunoassay. This has the advantage of allowing undesirable contamination to be ruled out.

The use according to the invention further includes in steps (b) to (f) the inserting of the at least one sample strip into the device, the transferring of the sample from the at least one cavity (i) into a cavity (ii) and optionally of a control substance from the at least one cavity (iv) into a further cavity (ii), the incubating of the sample and optionally of a control substance in the cavity/cavities (ii), the washing of the cavity/cavities (ii) with washing and/or rinsing solution stored in the device, the transferring of detecting reagent stored in the sample strip from the at least one cavity (iii) into the cavity/cavities (ii), and also the detecting of the biologically active substance to be detected. The device according to the invention carries out all of these steps, except for the inserting of the at least one sample strip, in a fully automatic manner. The sample and optionally control substance are preferably incubated in the cavity/cavities (ii) for from 1 to 30 minutes, more preferably for 1 to 15 minutes. The detecting of the biologically active substance to be detected is also carried out over a period of time of from 1 to 15, preferably from 1 to 5 minutes.

The use according to the invention preferably includes all of the reaction steps conventionally required for carrying out an immunoassay. It is preferred in this case for all of the reaction steps to be carried out within a period of time of 60, preferably 45 minutes, the rotary table being fully loaded.

In a particularly preferred embodiment, the biologically active substance is an antigen or antibody.

The present invention further relates to a sample strip according to the invention. A preferred embodiment of the present invention provides, for any desired individual immunoassay to be carried out using the device according to the invention, a specific sample strip which is sealed with a film and in which the at least one cavity (ii) is already coated with a suitable binding partner and in which the at least one cavity (iii) and optionally the at least one cavity (iv) is already loaded with the reaction reagent(s) required for the individual immunoassay to be carried out and optionally with a control substance.

The present invention further relates to the use of the sample strip according to the invention for carrying out an individual immunoassay in order to detect the presence of a biologically active substance in a sample. Preferably, the use is carried out in a device according to the invention for carrying out an individual immunoassay in order to detect the presence of a biologically active substance in a sample. More preferably, the biologically active substance is an antigen or an antibody.

The present invention further relates to a kit for carrying out an individual immunoassay in order to detect the presence of a biologically active substance in a sample, comprising a sample strip according to the invention. The kit is preferably used for carrying out an individual immunoassay in order to detect the presence of an antigen or antibody in a sample.

Finally, the invention further relates to the use of a kit according to the invention for carrying out an individual immunoassay in order to detect the presence of a biologically active substance in a sample. The use is preferably carried out in a device according to the invention for carrying out an individual immunoassay in order to detect a presence of a biologically active substance in a sample.

Furthermore, the invention will be illustrated in greater detail by the following figures.

### BRIEF DESCRIPTION OF THE DRAWINGS

FIG. 1 is a schematic illustration of a sample strip according to the invention comprising eight cavities occupied in an exemplary manner. Cavity No. 1 corresponds to a cavity (i), cavities Nos. 2 and 3 correspond to a cavity (ii), cavities Nos. 4 to 7 correspond to a cavity (iii) and cavity No. 8 corresponds to a cavity (iv).

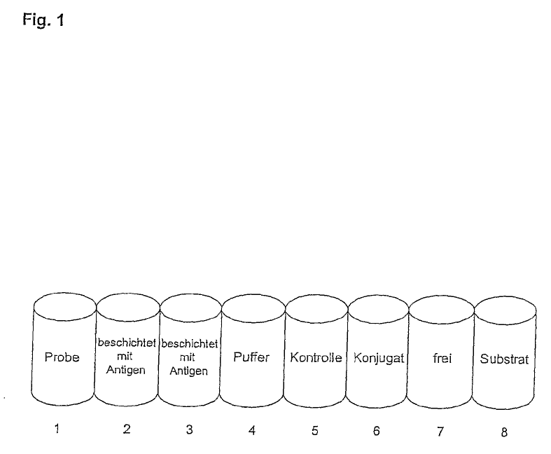

FIG. 2 is a schematic illustration of a sample strip according to the invention which is sealed with a film and comprises a bar code. The film has already been detached in the region of cavity No. 1 to allow introduction of the sample to be examined.

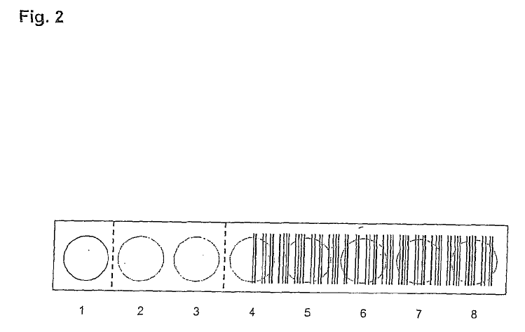

FIG. 3 is a schematic illustration of a means (a) of a device according to the invention, the transfer route of the sample strip to the different reaction stations of the individual immunoassay being indicated by the stations 1 to 6. Overall, the following reaction steps at the reaction stations are illustrated by way of example: 1: diluting the sample in cavity (i) with a diluting buffer from a cavity (iii) and pipetting it into a cavity (ii). Also pipetting the control substance from cavity (iv) into a further cavity (ii). 2: washing the two cavities (ii) with washing and/or rinsing solution stored in the device in order to remove non-bound constituents. 3: pipetting enzyme-labelled antigen/enzyme-labelled antibody from a further cavity (iii) (part 1 of the detection reagent) into the two cavities (ii). 4: washing the two cavities (ii) with washing and/or rinsing solution stored in the device in order to remove non-bound constituents. 5: pipetting the substrate from a further cavity (iii) tart 2 of the detection reagent) into the two cavities (ii). 6: photometric measuring of the enzyme/substrate reaction in both cavities (ii).

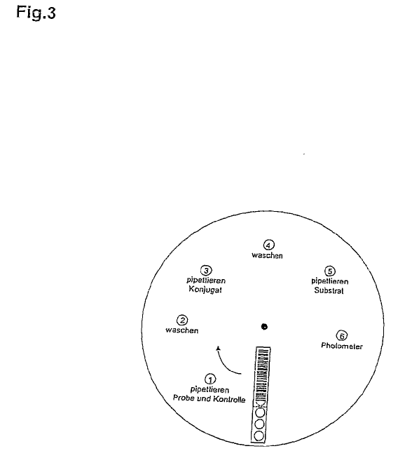

1. Device for carrying out an individual immunoassay in a fully automatic manner in order to detect the presence of a biologically active substance in a sample, comprising
(a) a means of receiving at least one sample strip, the sample strip comprising in an exclusively linear arrangement at least five cavities, of which at least one cavity (i) serves to provide the sample to be examined, at least one further cavity (ii) serves as a reaction chamber for carrying out the individual immunoassay, at least one further cavity (iii) serves as a reaction reagent reservoir for providing the reaction reagent(s) required for the individual immunoassay and optionally at least one further cavity (iv) serves to provide a control substance,
(b) a means for conveying the at least one sample strip along a transfer route to different reaction stations of the individual immunoassay, wherein the reaction stations comprise at least one decoding station, at least one pipetting station, at least one washing station and at least one detection station,
(c) at least one means for decoding a code specific to the individual immunoassay at the at least one decoding station,
(d) at least one pipetting system for carrying out the reaction steps of the individual immunoassay that are to be carried out at the at least one pipetting station and/or the at least one washing station,
(e) at least one reservoir for providing the washing and/or rinsing solution required at the at least one pipetting station and/or the at least one washing station,
(f) at least one means for evaluating the individual immunoassay at the at least one detection station, and
(g) at least one means for controlling the individual immunoassay in a fully automatic manner.

2. Device according to claim 1, characterised in that the biologically active substance is selected from an antibody, an antigen, a hapten, a hormone, a pharmacon, an opiate and a diagnostically important protein.

3. Device according to claim 1, characterised in that the biologically active substance is an autoimmune antibody.

4. Device according to claim 1, characterised in that the means (a) for receiving at least one sample strip is a rotary table.

5. Device according to claim 1, characterised in that rotary table receives up to 30 sample strips.

6. Device according to claim 1, characterised in that the at least one sample strip has in an exclusively linear arrangement eight cavities, of which one cavity (i) serves to provide the sample to be examined, two cavities (ii) serve as a reaction chamber for carrying out the individual immunoassay, four cavities (iii) serve as a reaction reagent reservoir for providing the reaction reagent(s) required for the individual immunoassay and optionally one cavity (iv) serves to provide a control substance.

7. Device according to claim 6, characterised in that the at least one cavity (ii) which serves as a reaction chamber for carrying out the individual immunoassay is coated with a suitable binding partner which, under the reaction conditions of the individual immunoassay, binds to the biologically active substance to be detected.

8. Device according to claim 6, characterised in that the at least one cavity (iii) contains all of the reaction reagents required for the individual immunoassay except for a washing and/or rinsing buffer.

9. Device according to claim 8, characterised in that the reaction reagents required for the individual immunoassay are selected from a buffer for diluting the sample to be examined and optionally the control substance and at least one detection reagent for the detection reaction.

10. Device according to claim 9, characterised in that the detection reagent comprises an enzyme-labelled antigen or an enzyme-labelled antibody and a suitable substrate which is converted by the enzyme of the enzyme-labelled antigen or antibody and can be detected photometrically or fluorimetrically.

11. Device according to claim 6, characterised in that the at least one cavity (iv) contains a control substance.

12. Device according to claim 6, characterised in that the sample strip is sealed with a film.

13. Device according to claim 12, characterised in that the film has perforations, so the film can easily be removed via the cavities (i) and/or (ii) before the sample strip is received in the means (a).

14. Device according to claim 12, characterised in that the film has a code specific to the individual immunoassay.

15. Device according to claim 14, characterised in that the code specific to the individual immunoassay is preferably located above the cavities (iii) and (iv).

16. Device according to claim 14, characterised in that the code specific to the individual immunoassay serves to detect the individual immunoassay to be carried out and/or to store the calibration curve of the individual immunoassay to be carried out.

17. Device according to claim 14, characterised in that the code specific to the individual immunoassay is a bar code.

18. Device according to claim 1, characterised in that the means (b) for conveying the at least one sample strip comprises a motor.

19. Device according to claim 1, characterised in that the at least one means (f) for evaluating the individual immunoassay is selected from a photometer, a fluorimeter and a computer with associated software.

20. Use of a device according to claim 1 for carrying out an individual immunoassay in a fully automatic manner for detecting an antibody in a sample, including the steps:
(a) preparing the at least one sample strip,
(b) inserting the at least one sample strip into the device,
(c) transferring the sample from the at least one cavity (i) into a cavity (ii) and optionally a control substance from the at least one cavity (iv) into a further cavity (ii),
(d) incubating the sample and optionally a control substance in the cavity/cavities (ii),
(e) washing the cavity/cavities (ii) with washing and/or rinsing solution stored in the device,
(f) transferring detection reagents stored in the sample strip from the at least one cavity (iii) into the cavity/cavities (ii), and
(g) detecting the biologically active substance to be detected.

21. Sample strip for carrying out an individual immunoassay for detecting a biologically active substance in a sample, characterised in that it displays the features described in claim 1.

22. Sample strip according to claim 21, characterised in that the biologically active substance is an antibody or antigen.

23. (canceled)

24. (canceled)

25. Kit for carrying out an individual immunoassay in order to detect the presence of a biologically active substance in a sample, characterised in that it comprises a sample strip according to claim 21.

26. Use of a kit according to claim 25 for carrying out an individual immunoassay in order to detect the presence of a biologically active substance in a sample.

27. (canceled)

28. A method for carrying out an individual immunoassay in order to detect the presence of a biologically active substance in a sample comprising using a sample strip of claim 21.

29. A method for carrying out an individual immunoassay in order to detect the presence of a biologically active substance in a sample comprising using a sample strip of claim 1.
